(19) United States
(12) Patent Application Publication
(10) Pub. No.: US 2019/0053710 A1
(43) Pub. Date: Feb. 21, 2019

(54) BIO-INFORMATION ESTIMATION APPARATUS, AND APPARATUS AND METHOD FOR SPECTRUM QUALITY ASSESSMENT

(71) Applicant: SAMSUNG ELECTRONICS CO., LTD., Suwon-si (KR)

(72) Inventors: June Young Lee, Seongnam-si (KR); Eui Seok Shin, Yongin-si (KR); Seung Jun Lee, Seoul (KR)

(73) Assignee: SAMSUNG ELECTRONICS CO., LTD., Suwon-si (KR)

(21) Appl. No.: 15/894,111

(22) Filed: Feb. 12, 2018

(30) Foreign Application Priority Data
Aug. 17, 2017 (KR) 10-2017-0104168

(51) Int. Cl. A61B 5/00

(52) U.S. Cl. CPC A61B 5/0075; A61B 5/441; A61B 5/7246; A61B 5/0077

(57) ABSTRACT

A spectrum quality assessment apparatus includes a spectrum obtainer configured to obtain a skin spectrum; and a processor configured to assess a quality of the obtained skin spectrum based on at least one of a light intensity of the obtained skin spectrum, an absorbance with respect to a wavelength of the obtained skin spectrum, a spectrum reconstructed based on a skin composition spectrum, and a similarity between a reference skin spectrum and the obtained skin spectrum.

### CROSS-REFERENCE TO RELATED APPLICATION(S)

This application claims priority from Korean Patent Application No. 10-2017-0104168, filed on 17 Aug. 2017, in the Korean Intellectual Property Office, the entire disclosure of which is incorporated herein by reference for all purposes.

### BACKGROUND

### 1. Field

Apparatuses and methods consistent with exemplary embodiments relate to technology for assessing the quality of a spectrum obtained from a subject and estimating bio-information, and more particularly to a bio-information estimation apparatus, and an apparatus and a method for spectrum quality assessment.

### 2. Description of the Related Art

An optical method may be utilized to detect biomolecules in a non-invasive manner. Light absorbance measurement methods may be divided according to optical wavelengths such as near infrared (NIR), mid infrared (MIR), and the like, and light scattering methods may be divided into an elastic scattering method and an inelastic scattering method.

Among the methods, a method of measuring absorbance of a near-infrared wavelength employs light absorption characteristics which vary depending on the wavelengths of biomolecules. For example, the method of measuring absorbance of the near-infrared wavelength may be used to measure main components, such as blood glucose, cholesterol, collagen, elastin, keratin, and the like, with very high specificity and reproducibility.

### SUMMARY

In an aspect according to an exemplary embodiment, there is provided a spectrum quality assessment apparatus, including: a spectrum obtainer configured to obtain a skin spectrum; and a processor configured to assess a quality of the obtained skin spectrum based on at least one of a light intensity of the obtained skin spectrum, an absorbance with respect to a wavelength of the obtained skin spectrum, a spectrum reconstructed based on a skin composition spectrum, and a similarity between a reference skin spectrum and the obtained skin spectrum.

The obtained skin spectrum may include a skin near-infrared absorption spectrum.

In response to the light intensity in at least one from among a wavelength region of 4000 cm⁻¹ to 4150 cm⁻¹ and a wavelength region of 4960 cm⁻¹ to 5280 cm⁻¹ of the obtained skin spectrum exceeding a predetermined threshold, the processor may determine that the obtained skin spectrum is distorted.

The processor may obtain a water path length based on a change in the absorbance with respect to the wavelength of the obtained skin spectrum and a reference absorbance with respect to a wavelength of a spectrum predetermined according to a center distance between a light source and a light detector, and in response to the obtained water path length being less than a predetermined value, the processor may determine that the obtained skin spectrum is distorted.

The processor may reconstruct the spectrum based on the skin composition spectrum, and in response to a difference in the absorbance between the obtained skin spectrum and the reconstructed spectrum exceeding a predetermined threshold, the processor may determine that the obtained skin spectrum is distorted.

The processor may generate the reconstructed spectrum by using a Classical Least square (CLS) algorithm.

The processor may obtain the similarity between the obtained skin spectrum and the reference skin spectrum according to a change of the absorbance with respect to the wavelength, and in response to the similarity being less than a predetermined threshold, the processor may determine that the obtained skin spectrum is distorted.

The spectrum obtainer may include a light source configured to emit light to a subject, and a light detector configured to detect light reflected or scattered from the subject.

The apparatus may further include an output part configured to output at least one from among a result of quality assessment of the obtained skin spectrum and guide information to correct distortion of the obtained skin spectrum.

In an aspect according to another exemplary embodiment, there is provided a bio-information estimation apparatus, including: a spectrum obtainer including a light source configured to emit light to a subject, and a light detector configured to detect light reflected or scattered from the subject; and a processor configured to determine a degree of distortion of an obtained skin spectrum, and to estimate bio-information based on a valid skin spectrum determined according to the degree of distortion.

The processor may determine the degree of distortion of the obtained skin spectrum based on at least one from among a light intensity of the obtained skin spectrum, an absorbance with respect to a wavelength, a spectrum reconstructed based on a skin composition spectrum, and a similarity between a reference skin spectrum and the obtained skin spectrum.

In response to determining that the obtained skin spectrum is not valid according to the degree of distortion, the processor may control the spectrum obtainer to re-obtain a skin spectrum.

The processor may generate guide information to correct at least one from among a state of a contact between the spectrum obtainer and the subject and a measurement position of the spectrum obtainer with respect to the subject according to the degree of distortion of the obtained skin spectrum.

The processor may estimate bio-information by using a bio-information correlation model pre-generated based on the valid skin spectrum.

In an aspect according to still another exemplary embodiment, there is provided a spectrum quality assessment method, including: obtaining a skin spectrum; and assessing a quality of the obtained skin spectrum based on at least one from among a light intensity of the obtained skin spectrum, an absorbance with respect to a wavelength of the obtained skin spectrum, a spectrum reconstructed based on a skin composition spectrum, and a similarity between a reference skin spectrum and the obtained skin spectrum.

The obtained skin spectrum may include a skin near-infrared absorption spectrum.

The assessing may include determining that the obtained skin spectrum is distorted in response to the light intensity in at least one from among a wavelength region of 4000 cm⁻¹ to 4150 cm⁻¹ a wavelength region of 4960 cm⁻¹ to 5280 cm⁻¹ of the obtained skin spectrum exceeding a predetermined threshold.

The assessing may include obtaining a water path length based on a change in the absorbance with respect to the wavelength of the obtained skin spectrum and a reference absorbance with respect to a wavelength of a spectrum predetermined according to a center distance between a light source and a light detector; and in response to the water path length being less than a predetermined value, determining that the obtained skin spectrum is distorted.

The assessing may include generating the spectrum reconstructed based on the skin composition spectrum; and in response to a difference in the absorbance between the obtained skin spectrum and the reconstructed spectrum exceeding a predetermined threshold, determining that the obtained skin spectrum is distorted.

The assessing may include obtaining the similarity between the obtained skin spectrum and the reference skin spectrum according to a change of the absorbance with respect to the wavelength; and in response to the similarity being less than a predetermined threshold, determining that the obtained skin spectrum is distorted.

### BRIEF DESCRIPTION OF THE DRAWINGS

The above and/or other aspects will become apparent and more readily appreciated from the following description of the exemplary embodiments, taken in conjunction with the accompanying drawings.

FIG. 1 is a block diagram illustrating an example of a spectrum quality assessment apparatus according to an exemplary embodiment.

FIG. 2 is a diagram for explaining an example of assessing a quality of a skin spectrum based on a light intensity in a specific wavelength region of an obtained skin spectrum according to an exemplary embodiment.

FIG. 3 is a diagram for explaining an example of assessing a quality of a skin spectrum based on absorbance for a wavelength according to an exemplary embodiment.

FIG. 4 is a diagram for explaining an example of assessing a quality of a skin spectrum based on a reconstructed spectrum according to an exemplary embodiment.

FIG. 5 is a diagram for explaining an example of assessing a quality of a skin spectrum based on similarity between a reference skin spectrum and an obtained skin spectrum according to an exemplary embodiment.

FIG. 6 is a block diagram illustrating an example of a spectrum quality assessment apparatus according to another exemplary embodiment.

FIG. 7 is a block diagram illustrating an example of a bio-information estimation apparatus according to an exemplary embodiment.

FIG. 8 is a flowchart illustrating an example of a spectrum quality assessment method according to an exemplary embodiment.

FIG. 9 is a flowchart illustrating an example of a spectrum quality assessment method according to another exemplary embodiment.

FIG. 10 is a flowchart illustrating an example of a bio-information estimation method according to an exemplary embodiment.

### DETAILED DESCRIPTION

Hereinafter, embodiments of the disclosure will be described in detail with reference to the accompanying drawings. It should be noted that, in the drawings, the same reference symbols refer to same parts although illustrated in other drawings. In the following description, a detailed description of known functions and configurations incorporated herein will be omitted when it may obscure the subject matter of the present invention. Throughout the drawings and the detailed description, unless otherwise described, the same drawing reference numerals will be understood to refer to the same elements, features, and structures. The relative size and depiction of these elements may be exaggerated for clarity, illustration, and convenience.

Process steps described herein may be performed differently from a specified order, unless a specified order is clearly stated in the context of the disclosure. That is, each step may be performed in a specified order, at substantially the same time, or in a reverse order.

Further, the terms used throughout this specification are defined in consideration of the functions according to exemplary embodiments, and can be varied according to a purpose of a user or manager, or precedent and so on. Therefore, definitions of the terms should be made on the basis of the overall context.

Any references to singular may include plural unless expressly stated otherwise. In the present specification, it should be understood that the terms, such as ‘including’ and ‘having,’ etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Hereinafter, a bias-information estimation apparatus, and an apparatus and a method for assessing the quality of a spectrum according to exemplary embodiments will be described below with the accompanying drawings.

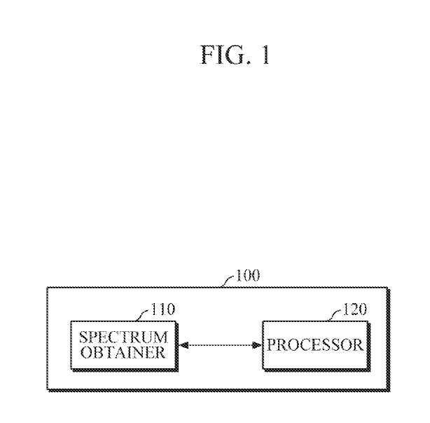

FIG. 1 is a block diagram illustrating an example of a spectrum quality assessment apparatus according to an exemplary embodiment. The spectrum quality assessment apparatus 100 may assess the quality of an obtained skin spectrum by analyzing absorbance or light intensity of the obtained skin spectrum.

For example, the spectrum quality assessment apparatus 100 may obtain the skin spectrum by emitting light to a user's skin and detecting light which is reflected or scattered therefrom, and may analyze the absorbance or light intensity in a specific wavelength region of the obtained skin spectrum. In this case, the spectrum quality assessment apparatus 100 may assess the quality of the obtained skin spectrum by determining whether the skin spectrum is obtained by coming into hard contact with a subject or whether distortion occurs due to Fresnel reflection or by stray light contamination.

The spectrum quality assessment apparatus 100 may be implemented as a software module or may be manufactured in the form of a hardware chip to be embedded in various types of electronic apparatuses. Examples of the electronic apparatuses may include a cellular phone, a smartphone, a tablet PC, a laptop computer, a personal digital assistant (PDA), a portable multimedia player (PMP), a navigation, an MP3 player, a digital camera, a wearable device, and the like. However, the electronic apparatuses are not limited to these examples, and examples thereof may include various apparatuses.

Referring to FIG. 1, the spectrum quality assessment apparatus 100 includes a spectrum obtainer 110 and a processor 120. Here, the processor 120 may include one or more processors, a memory, and a combination thereof.

The spectrum obtainer 110 may obtain a user's skin spectrum.

In an exemplary embodiment, the skin spectrum may be a skin near-infrared absorption spectrum which is measured by emitting near-infrared ray to a user's skin. However, the skin spectrum is not limited thereto, and may be a skin near-infrared transmission spectrum or a skin near-infrared reflectance spectrum.

The spectrum obtainer 110 may include a light source which emits light to a subject, and a light detector which detects light reflected or scattered from the subject. The spectrum obtainer 110 may directly generate skin spectrum data by using the light detected by the light detector.

Further, the spectrum obtainer 110 may communicate with an external device to receive skin spectrum data of a user from the external device. For example, the spectrum obtainer 110 may receive the skin spectrum data of the user from the external device by using various communication methods such as Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra Wideband (UWB) communication, Ant+ communication, WIFI communication, Radio Frequency Identification (RFID) communication, and the like. However, this is merely exemplary and the communication is not limited thereto.

Further, examples of the external device may include a cellular phone, a smartphone, a tablet PC, a laptop computer, a personal digital assistant (PDA), a portable multimedia player (PMP), a navigation, an MP3 player, a digital camera, a wearable device, and the like. However, the external device is not limited thereto, and examples thereof may include various devices that may store the user's skin spectrum data.

In an exemplary embodiment, the processor 120 may assess the quality of the obtained skin spectrum by analyzing the obtained skin spectrum and determining a degree of distortion thereof.

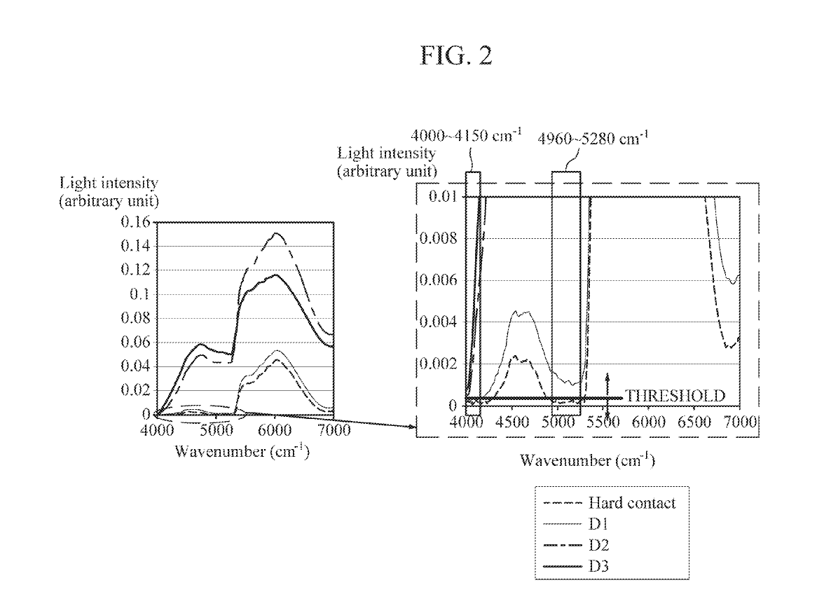

For example, the processor 120 may assess the quality of the obtained skin spectrum based on at least one from among the following: a light intensity of the obtained skin spectrum in a specific wavelength region; absorbance for a wavelength; a spectrum reconstructed based on a skin composition spectrum; and similarity between a reference skin spectrum and the obtained skin spectrum, which will be described below in detail with reference to FIGS. 2 to 5.

FIGS. 2 to 5 are diagrams for explaining an example of assessing a quality of a skin spectrum by determining a degree of distortion of an obtained skin spectrum by a spectrum quality assessment apparatus 100 according to exemplary embodiments.

FIG. 2 is a diagram for explaining an example of assessing a quality of a skin spectrum based on a light intensity in a specific wavelength region of an obtained skin spectrum according to an exemplary embodiment. FIG. 2 illustrates a wavelength-light intensity graph (indicated by a dotted line) showing an example where a light source and/or a light detector is in hard contact with a subject, and wavelength-light intensity graphs D1, D2, and D3 showing examples where the light source and/or the light detector is not in normal contact with the subject.

In an exemplary embodiment, the processor 120 may calculate a change in the light intensity with respect to optical wavelengths by analyzing the obtained skin spectrum, and may determine a degree of distortion of the obtained skin spectrum by comparing the light intensity in a specific wavelength region with a threshold.

For example, with respect to aspects of the change in the light intensity with respect to optical wavelengths, a skin spectrum obtained in the case where the light source and/or the light detector is in hard contact with the subject may be different from a skin spectrum obtained in the case where the light source and/or the light detector is not in normal contact with the subject.

For example, in the case where the light source and/or the light detector is in normal contact with the subject to obtain a skin spectrum, the light intensity in a wavelength region (or wavenumber) of 4000 cm⁻¹ or 5000 cm⁻¹ is close to about 0 (arbitrary unit). In this case, the processor 120 may determine a degree of distortion of the obtained skin spectrum by analyzing the light intensity in the wavelength region and by determining whether the analyzed light intensity exceeds a predetermined threshold.

For example, the processor 120 may assess the quality of the obtained skin spectrum in such a manner that, in the case where the light intensity in the wavelength region (or wavenumber) of 4000 cm⁻¹ to 4150 cm⁻¹ and/or 4960 cm⁻¹ to 5280 cm⁻¹ of the obtained skin spectrum exceeds the predetermined threshold, the processor 120 may determine that the obtained skin spectrum is distorted, and in the case where the light intensity in that wavelength region is equal to or less than the predetermined threshold, the processor 120 may determine that the obtained skin spectrum is of good quality.

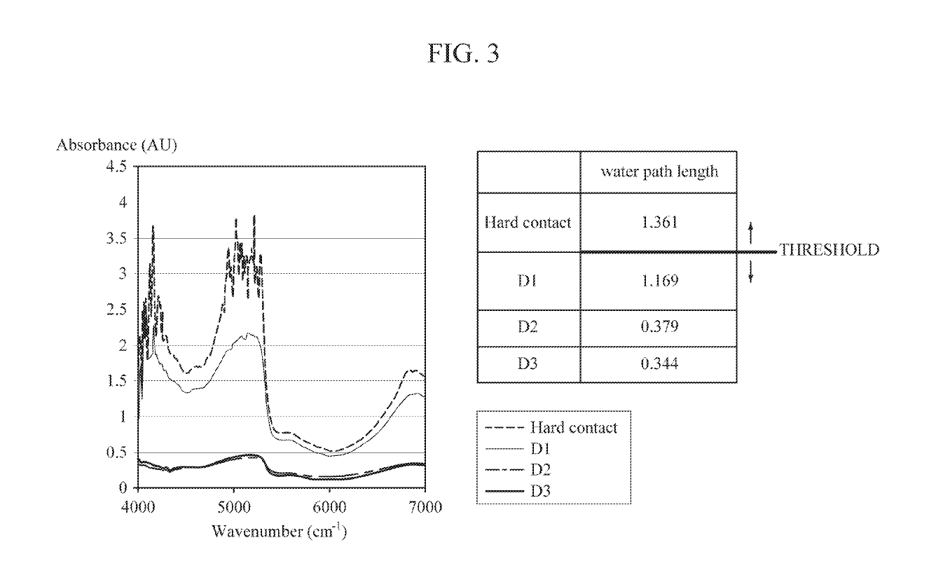

FIG. 3 is an exemplary diagram for explaining an example of assessing a quality of a skin spectrum based on absorbance for a wavelength according to an exemplary embodiment.

FIG. 3 illustrates a wavelength-absorbance graph (indicated by a dotted line) showing an example where a light source and/or a light detector is in hard contact with a subject, and wavelength-absorbance graphs D1, D2, and D3 showing examples where the light source and/or the light detector is not in normal contact with the subject.

In an exemplary embodiment, the processor 120 may calculate a water path length based on a change in absorbance for a wavelength of the obtained skin spectrum and may determine whether an obtained skin spectrum is distorted by comparing the calculated water path length with a reference water path length.

For example, referring to FIG. 3, the wavelength-absorbance graph, showing an example where the light source and/or the light detector is in hard contact with a subject, represents a quadratic function which is convex downward in the region (or wavenumber) of about 4000 cm⁻¹ to 5000 cm⁻¹ and the region (or wavenumber) of about 5200 cm⁻¹ to 7000 cm⁻¹. In this case, the processor 120 may calculate the water path length by comparing the shape of the wavelength-absorbance graph with a wavelength-absorbance graph for the reference water path length, and may determine whether the obtained skin spectrum is distorted by comparing the calculated water path length with the reference water path length under the same condition.

Here, the water path length may refer to a length of a path of light traveling until the light reaches the light detector after being emitted from the light source and transmitting through the subject. Further, the reference water path length may be predetermined based on a change of absorbance for a wavelength, and may be pre-generated into one or more groups according to at least one criterion among a center distance between the light source and the light detector, a diameter of the light source and/or the light detector, a composition of a subject, and reflectance or absorbance on the surface of the subject.

For example, the reference water path length may be pre-generated in such a manner that a wavelength-absorbance graph is obtained by using a spectrum obtaining apparatus (e.g., spectrograph) which is disposed at a user's wrist with the light source and the light detector, having a diameter of about 0.2 mm, being spaced apart from each other by a center distance of about 0.6 mm, and then the water path length is determined to be about 1.3 mm under that measurement condition. In this case, the processor 120 may calculate the water path length from the wavelength-absorbance graph measured under the same condition, and may determine whether the obtained skin spectrum is distorted by comparing the calculated water path length with the reference water path length (e.g., about 1.3 mm).

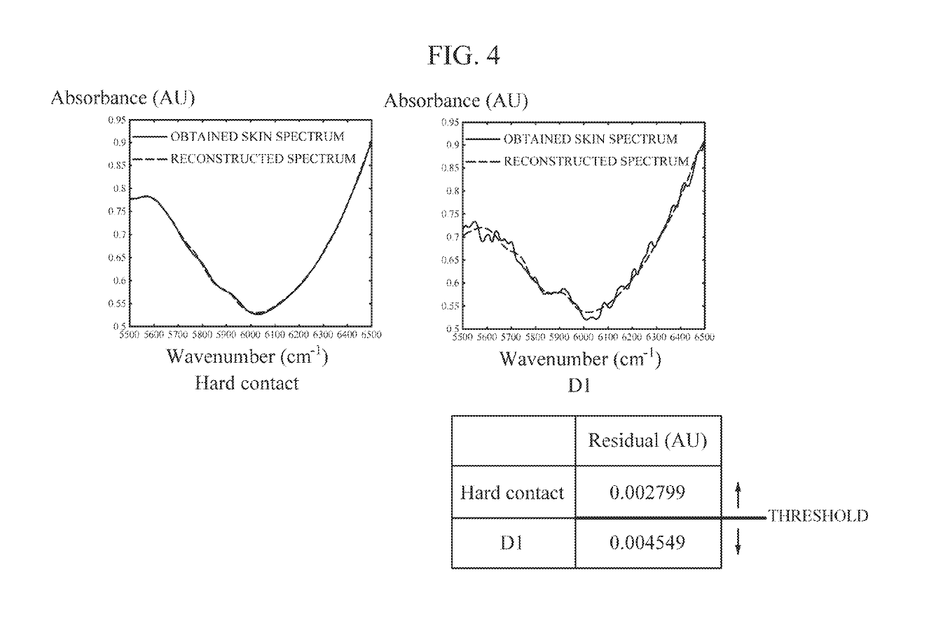

FIG. 4 is a diagram for explaining an example of assessing a quality of a skin spectrum based on a reconstructed spectrum according to an exemplary embodiment.

FIG. 4 illustrates a wavelength-absorbance graph and a graph showing a spectrum reconstructed based on a skin composition in an example where a light source and/or a light detector is in hard contact with a subject, and a wavelength-absorbance graph D1 and a graph showing the spectrum reconstructed based on the skin composition in an example where the light source and/or the light detector is not in hard contact with the subject.

In an exemplary embodiment, the processor 120 may reconstruct the spectrum based on a skin composition spectrum, and in the case where a difference in absorbance between the obtained skin spectrum and the reconstructed spectrum exceeds a predetermined threshold, the processor 120 may determine that the obtained skin spectrum is distorted.

Here, the reconstructed spectrum is a spectrum generated by combining spectrums of a main skin composition (e.g., lipid, protein, moisture, etc.), and may be a spectrum in which distortion factors, such as noise introduced from the outside, Fresnel reflection between the light source the light detector, stray light contamination, or the like, are not included.

For example, the processor 120 may calculate a residual by comparing the obtained skin spectrum with the reconstructed spectrum, and in the case where the calculated residual exceeds a predetermined threshold, the processor 120 may determine that the obtained skin spectrum is distorted.

For example, referring to FIG. 4, the processor 120 may calculate the residual by adding up areas of a closed curve generated by intersection between the wavelength-absorbance graph and the graph showing the spectrum reconstructed based on the skin composition in an example where the light source and/or the light detector is in hard contact with the subject and/or a closed curve generated by intersection between the wavelength-absorbance graph D1 and the graph showing the spectrum reconstructed based on the skin composition in an example where the light source and/or the light detector is not in hard contact with the subject, and the processor 120 may determine whether the obtained skin spectrum is distorted by comparing the calculated residual with a predetermined threshold.

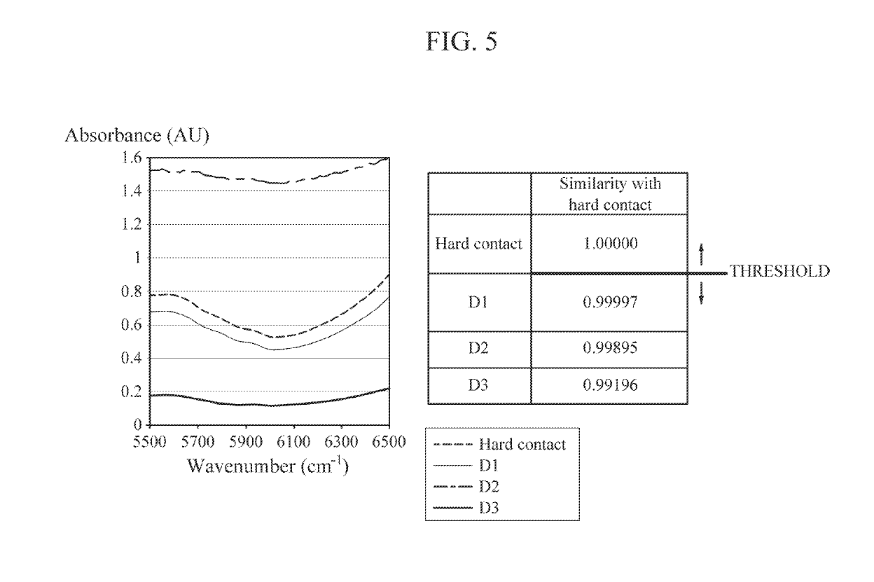

FIG. 5 is a diagram for explaining an example of assessing a quality of a skin spectrum based on similarity between a reference skin spectrum and an obtained skin spectrum according to an exemplary embodiment. FIG. 5 illustrates a wavelength-absorbance graph (indicated by a dotted line) showing an example where a light source and/or a light detector is in hard contact with a subject, and wavelength-absorbance graphs D1, D2, and D3 showing examples where the light source and/or the light detector is not in normal contact with the subject.

In an exemplary embodiment, the processor 120 may determine a degree of distortion of the obtained skin spectrum based on similarity between the obtained skin spectrum and the reference skin spectrum.

Here, the reference skin spectrum is a spectrum to be used for comparison with the obtained skin spectrum, and may be a skin spectrum which is pre-obtained based on at least one of a user's age, gender, race, and measured part while the light source and/or the light detector is in hard contact with a subject.

For example, referring to FIG. 5, the processor 120 may calculate cosine similarity between the obtained skin spectrum and the reference skin spectrum. For example, the processor 120 may calculate the cosine similarity between the obtained skin spectrum and the reference skin spectrum by extracting a vector array for calculation of the cosine similarity between the obtained skin spectrum and the reference skin spectrum in the same specific wavelength region, and by using an inner product of the extracted vector; and in the case where the calculated similarity is less than a predetermined threshold, the processor 120 may determine that the obtained skin spectrum is distorted. However, the calculation of the cosine similarity is not limited thereto, and the processor 120 may calculate the cosine similarity based on a similarity determination algorithm such as Hit Quality Index and Euclidean distance.

As described above with reference to FIGS. 2 to 5, the processor 120 may determine a degree of distortion of the skin spectrum obtained based on a light intensity in a specific wavelength region of the obtained skin spectrum, absorbance for a wavelength, a spectrum reconstructed based on a skin composition spectrum, and similarity between the reference skin spectrum and the obtained skin spectrum. For convenience of explanation, various exemplary embodiments are described separately, but it should be construed that the exemplary embodiments may be performed individually, sequentially, and/or in parallel.

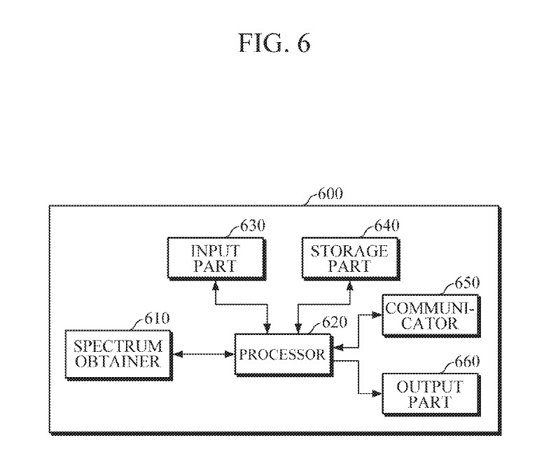

FIG. 6 is a block diagram illustrating an example of a spectrum quality assessment apparatus according to another exemplary embodiment.

Referring to FIG. 6, a spectrum quality assessment apparatus 600 includes a spectrum obtainer 610, a processor 620, an input part 630, a storage part 640, a communicator 650, and an output part 660. Here, the spectrum obtainer 610 and the processor 620 may perform the same or similar functions as those of the spectrum obtainer 110 and the processor 120 described above with reference to FIG. 1, and repetitive descriptions are omitted.

The input part 630 may receive an input of various operation signals and/or data to be used for assessment of a spectrum quality from a user. In an exemplary embodiment, the input part 630 may include, for example but not limited to, a keypad, a dome switch, a touch pad (static pressure/capacitance), a jog wheel, a jog switch, a hardware (H/W) button, and the like. Particularly, the touch pad, which forms a layer structure with a display, may be called a touch screen.

For example, the input part 630 may receive an input of user feature information including one or more of race, age, gender, stature, weight, and health information of users.

For example, based on the input information, the processor 620 may select a skin composition spectrum for generating a reconstructed spectrum, or a reference spectrum.

The storage part 640 may store programs or commands for operation of the spectrum quality assessment apparatus 600, and may store data input to and output from the spectrum quality assessment apparatus 600. For example, the storage part 640 may store the user feature information input through the input part 630, data of the skin spectrum obtained by the spectrum obtainer 610, the reference spectrum, the skin composition spectrum, and the like.

The storage part 640 may include, for example but not limited to, at least one storage medium from among a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like. Further, the spectrum quality assessment apparatus 600 may operate an external storage medium, such as web storage and the like, which performs a storage function of the storage part 640 on the Internet.

The communicator 650 may perform communication with an external device. For example, the communicator 650 may transmit, to the external device, the user feature information input through the input part 630, data of the skin spectrum obtained by the spectrum obtainer 610, a result of assessment of a spectrum quality by the processor 620, and the like; or may receive various data, such as the user feature information, the skin spectrum, the reference spectrum, the skin composition spectrum, or the like, from the external device.

In this case, the external device may be medical equipment using a database (DB) of the spectrum quality and/or the result of assessment of the spectrum quality, a printer to print out results, or a display to display the result of assessment of the spectrum quality. In addition, the external device may be a digital TV, a desktop computer, a cellular phone, a smartphone, a tablet PC, a laptop computer, a personal digital assistant (PDA), a portable multimedia player (PMP), a navigation, an MP3 player, a digital camera, a wearable device, and the like, but is not limited thereto.

The communicator 650 may communicate with external devices by using various communication methods such as Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra Wideband (UWB) communication, Ant+ communication, WIFI communication, Radio Frequency Identification (RFID) communication, 3G communication, 4G communication, 5G communication, and the like. However, this is merely exemplary and the communication is not limited thereto.

The output part 660 may output at least one or more of the result of assessment of the spectrum quality, a guide (or guide information) for correcting distortion of the obtained skin spectrum, and warning information, by control of the processor 620.

In an exemplary embodiment, the output part 660 may output at least one or more of the result of assessment of the spectrum quality, the guide for correcting distortion of the obtained skin spectrum, and the warning information, by using at least one of an acoustic method, a visual method, and a tactile method. To this end, the output part 660 may include a display, a speaker, a vibrator, and the like.

For example, upon determining that the obtained skin spectrum is distorted based on the result of assessment of the spectrum quality, the processor 620 may output an alarm for re-measuring a spectrum through the output part 660, or may generate a guide for correcting at least one of a contact state (e.g., a state of a contact between the spectrum obtainer 610 and the subject) and a measurement position of the spectrum obtainer 610 with respect to the subject.

Further, upon determining that a spectrum is to be re-measured according to a degree of distortion of the obtained skin spectrum, the processor 620 may control the spectrum obtainer 610 to re-measure the spectrum. However, the processor 620 is not limited thereto, and the processor 620 may receive a new skin spectrum from an external skin spectrum database (DB).

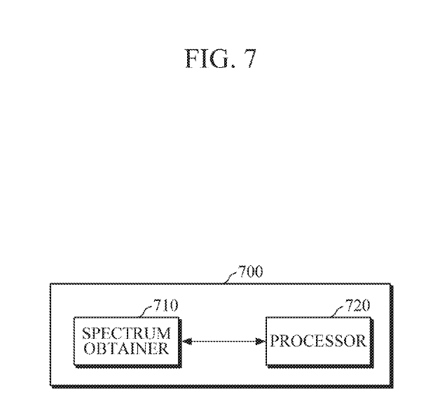

FIG. 7 is a block diagram illustrating an example of a bio-information estimation apparatus according to an exemplary embodiment.

Referring to FIG. 7, the bio-information estimation apparatus 700 includes a spectrum obtainer 710 and a processor 720.

The spectrum obtainer 710 may obtain a skin spectrum of a user.

For example, the spectrum obtainer 710 may include a light source which emits light to a subject, and a light detector which detects light reflected or scattered from the subject. The spectrum obtainer 710 may generate skin spectrum data by using the light detected by the light detector. However, the spectrum obtainer 710 is not limited thereto, and may obtain a skin spectrum from an external device or a spectrum database (DB).

The processor 720 may determine a degree of distortion of the obtained skin spectrum, and may estimate bio-information based on a valid skin spectrum determined according to a degree of distortion.

In an exemplary embodiment, the processor 720 may determine a degree of distortion of the obtained skin spectrum based on at least one from among the following: a light intensity of the obtained skin spectrum; absorbance for a wavelength; a spectrum reconstructed based on the obtained skin spectrum; and similarity between the reference skin spectrum and the obtained skin spectrum.

For example, upon determining the degree of distortion of the obtained skin spectrum, in the case where the degree of distortion is within a predetermined reference range, the processor 720 may determine that the skin spectrum is valid for estimating bio-information; and in the case where the degree of distortion exceeds the predetermined reference range, the processor 720 may determine that the skin spectrum is not valid for estimating bio-information, and may re-obtain spectrum data by controlling the spectrum obtainer 710.

The processor 720 may generate a guide (or guide information) for correcting at least one of a contact state and a measurement position of the spectrum obtainer 710 with respect to the subject according to the degree of distortion of the obtained skin spectrum, and may output the generated guide by using, for example, at least one from among an acoustic method, a visual method, and a tactile method.

In an exemplary embodiment, the processor 720 may estimate bio-information by using a bio-information correlation model pre-generated based on a valid skin spectrum.

Here, the bio-information may be skin components, such as moisture, protein, lipid, or various minerals, and may include at least one of blood glucose, cholesterol, and neutral fats as blood components.

Further, the bio-information correlation model may be a correlation model pre-generated by using a correlation between a biomaterial change and a spectrum change for a wavelength based on the obtained skin spectrum, and may be divided into user information, including a user's age, gender, weight, body mass index (BMI), and health information, and each measurement point of the skin spectrum.

For example, the bio-information correlation model may be a correlation model related to a change in near-infrared (NIR) spectrum data according to a change in blood glucose. The processor 720 may select an appropriate bio-information correlation model according to the types of bio-information to be measured, and may measure bio-information by comparing the selected bio-information correlation model with the obtained skin spectrum.

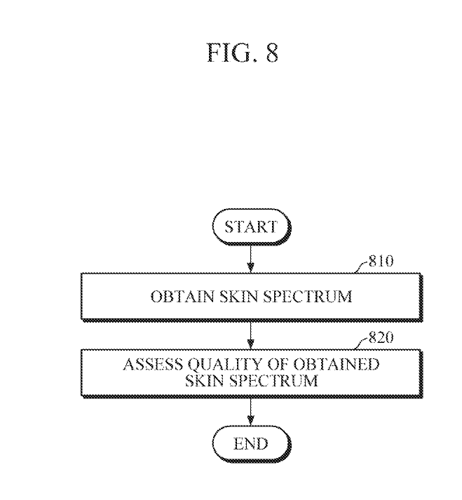

FIG. 8 is a flowchart illustrating an example of a spectrum quality assessment method according to an exemplary embodiment. The spectrum quality assessment method may be performed by any one of the spectrum quality assessment apparatuses 100 and 600 of FIGS. 1 and 6.

The spectrum quality assessment apparatus 100 may obtain a user's skin spectrum in 810.

In an exemplary embodiment, the skin spectrum may be a skin near-infrared absorption spectrum which is measured by emitting near-infrared ray to a user's skin. However, the skin spectrum is not limited thereto, and may be a skin near-infrared transmission spectrum or a skin near-infrared reflectance spectrum. Further, the spectrum quality assessment apparatus 100 may communicate with an external device to receive a user's skin spectrum data from the external device.

The spectrum quality assessment apparatus 100 may assess the quality of the obtained skin spectrum in 820 by analyzing the obtained skin spectrum and by determining a degree of distortion of the obtained skin spectrum.

For example, the spectrum quality assessment apparatus 100 may assess the quality of the obtained skin spectrum based on at least one from among the following: a light intensity of the obtained skin spectrum in a specific wavelength region; absorbance for a wavelength; a spectrum reconstructed based on a skin composition spectrum; and similarity between a reference skin spectrum and the obtained skin spectrum.

In an exemplary embodiment, the spectrum quality assessment apparatus 100 may calculate a change in the light intensity with respect to optical wavelengths by analyzing the obtained skin spectrum, and may determine a degree of distortion of the obtained skin spectrum by comparing the light intensity in a specific wavelength region with a threshold.

For example, with respect to aspects of the change in the light intensity for optical wavelengths, a skin spectrum obtained in the case where the light source and/or the light detector is in hard contact with the subject may be different from a skin spectrum obtained in the case where the light source and/or the light detector is not in normal contact with the subject.

For example, in the case where the light source and/or the light detector is in normal contact with the subject to obtain a skin spectrum, the light intensity in a wavelength region (or wavenumber) of 4000 cm⁻¹ or 5000 cm⁻¹ is close to about 0 (arbitrary unit). In this case, the spectrum quality assessment apparatus 100 may determine a degree of distortion of the obtained skin spectrum by analyzing the light intensity in the wavelength region and by determining whether the analyzed light intensity exceeds a predetermined threshold.

For example, the spectrum quality assessment apparatus 100 may assess the quality of the obtained skin spectrum in such a manner that, in the case where the light intensity in the wavelength region (or wavenumber) of 4000 cm⁻¹ to 4150 cm⁻¹ and/or 4960 cm⁻¹ to 5280 cm⁻¹ of the obtained skin spectrum exceeds the predetermined threshold, the spectrum quality assessment apparatus 100 may determine that the obtained skin spectrum is distorted; and in the case where the light intensity in that wavelength region is equal to or less than the predetermined threshold, the spectrum quality assessment apparatus 100 may determine that the obtained skin spectrum is of good quality.

In another example, the spectrum quality assessment apparatus 100 may calculate a water path length based on a change in absorbance for a wavelength of the obtained skin spectrum, and may determine whether an obtained skin spectrum is distorted by comparing the calculated water path length with a reference water path length.

The spectrum quality assessment apparatus 100 may calculate the wave path length by comparing the shape of the wavelength-absorbance graph with a wavelength-absorbance graph for the reference water path length, and may determine whether the obtained skin spectrum is distorted by comparing the calculated water path length with the reference water path length under the same condition.

For example, the reference water path length may be pre-generated in such a manner that a wavelength-absorbance graph is obtained by using a spectrum obtaining apparatus which is disposed at a user's wrist with the light source and the light detector, having a diameter of about 0.2 mm, being spaced apart from each other by a center distance of 0.6 mm, and then the water path length is determined to be about 1.3 mm under that measurement condition. In this case, the spectrum quality assessment apparatus 100 may calculate the water path length from the wavelength-absorbance graph measured under the same condition, and may determine whether the obtained skin spectrum is distorted by comparing the calculated water path length with the reference water path length (e.g., about 1.3 mm).

In another example, the spectrum quality assessment apparatus 100 may reconstruct the spectrum based on a skin composition spectrum; and in the case where a difference in absorbance between the obtained skin spectrum and the reconstructed spectrum exceeds a predetermined threshold, the spectrum quality assessment apparatus 100 may determine that the obtained skin spectrum is distorted.

Here, the reconstructed spectrum is a spectrum generated by combining spectrums of main skin components (e.g., lipid, protein, moisture, etc.), and may be a spectrum in which distortion factors, such as noise introduced from the outside, Fresnel reflection between the light source the light detector, stray light contamination, or the like, are not included.

For example, the spectrum quality assessment apparatus 100 may calculate a residual by comparing the obtained skin spectrum with the reconstructed spectrum; and in the case where the calculated residual exceeds a predetermined threshold, the spectrum quality assessment apparatus 100 may determine that the obtained skin spectrum is distorted.

For example, the spectrum quality assessment apparatus 100 may calculate the residual by adding up areas of a closed curve generated by intersection between the wavelength-absorbance graph and the graph showing the spectrum reconstructed based on the skin composition in an example where the light source and/or the light detector is in hard contact with the subject and/or a closed curve generated by intersection between the wavelength-absorbance graph D1 and the graph showing the spectrum reconstructed based on the skin composition in an example where the light source and/or the light detector is not in hard contact with the subject, and the spectrum quality assessment apparatus 100 may determine whether the obtained skin spectrum is distorted by comparing the calculated residual with a predetermined threshold.

In another example, the spectrum quality assessment apparatus 100 may determine a degree of distortion of the obtained skin spectrum based on similarity between the obtained skin spectrum and the reference skin spectrum.

Here, the reference skin spectrum is a spectrum to be used for comparison with the obtained skin spectrum, and may be a skin spectrum which is pre-obtained based on at least one of a user's age, gender, race, and measured part while the light source and/or the light detector is in hard contact with a subject.

For example, the spectrum quality assessment apparatus 100 may calculate cosine similarity between the obtained skin spectrum and the reference skin spectrum. For example, the spectrum quality assessment apparatus 100 may calculate the cosine similarity between the obtained skin spectrum and the reference skin spectrum by extracting a vector array for calculation of the cosine similarity between the obtained skin spectrum and the reference skin spectrum in the same specific wavelength region, and by using an inner product of the extracted vector; and in the case where the calculated similarity is less than a predetermined threshold, the spectrum quality assessment apparatus 100 may determine that the obtained skin spectrum is distorted.

However, the calculation of the cosine similarity is not limited thereto, and the spectrum quality assessment apparatus 100 may calculate the cosine similarity based on a similarity determination algorithm such as Hit Quality Index and Euclidean distance.

As described above with reference to various exemplary embodiments, the spectrum quality assessment apparatus 100 may determine the degree of distortion of the obtained skin spectrum based on a light intensity of the obtained skin spectrum in a specific wavelength region; absorbance for a wavelength; a spectrum reconstructed based on a skin composition spectrum; and similarity between the reference skin spectrum and the obtained skin spectrum. For convenience of explanation, various exemplary embodiments are described separately, but it should be construed that the embodiments may be performed individually, sequentially, and/or in parallel.

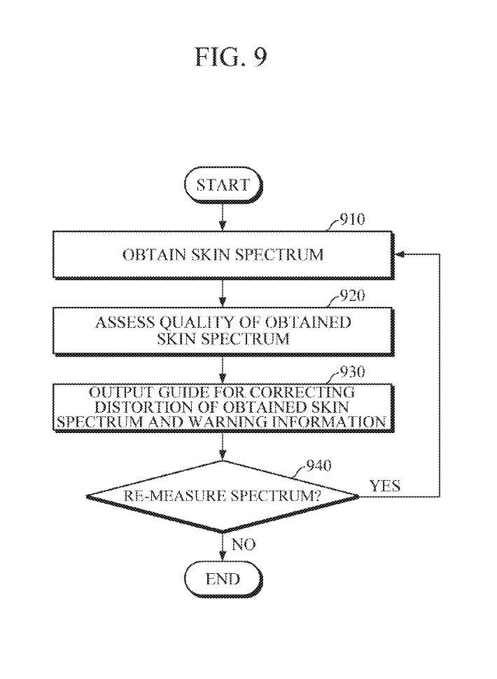

FIG. 9 is a flowchart illustrating an example of a spectrum quality assessment method according to another exemplary embodiment. The spectrum quality assessment method of FIG. 9 may be performed by the spectrum quality assessment apparatus 600 of FIG. 6.

The spectrum quality assessment apparatus 600 may obtain a user's skin spectrum in 910.

The spectrum quality assessment apparatus 600 may assess the quality of the obtained skin spectrum by analyzing the obtained skin spectrum and by determining a degree of distortion of the obtained skin spectrum in 920.

Further, based on a result of the quality assessment of the obtained skin spectrum, the spectrum quality assessment apparatus 600 may output at least one or more of a guide (or guide information) for correcting distortion of the obtained skin spectrum and warning information in 930.

For example, the spectrum quality assessment apparatus 600 may output at least one or more of the guide for correcting distortion of the obtained skin spectrum and the warning information by using, for example but not limited to, at least one of an acoustic method, a visual method, and a tactile method.

In addition, upon determining that a spectrum is to be re-measured according to a degree of distortion of the obtained skin spectrum, the spectrum quality assessment apparatus 600 may re-measure the spectrum in 940. For example, the spectrum quality assessment apparatus 600 may re-measure the spectrum of a subject, and may receive a new skin spectrum from an external skin spectrum database (DB).

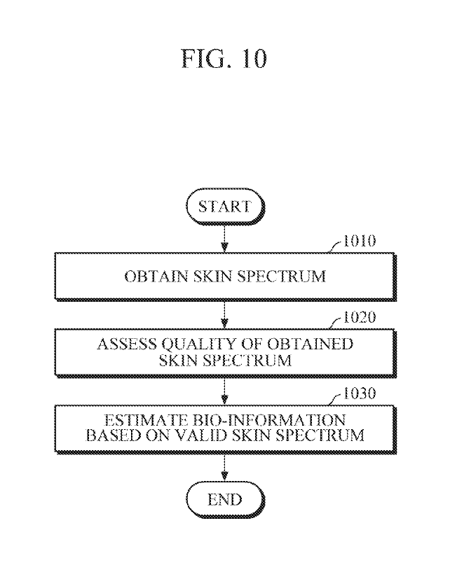

FIG. 10 is a flowchart illustrating an example of a bio-information estimation method according to an exemplary embodiment. The bio-information estimation method of FIG. 10 may be performed by the bio-information estimation apparatus 700.

The bio-information estimation apparatus 700 may obtain a user's skin spectrum in 1010.

The bio-information estimation apparatus 700 may assess the quality of the obtained skin spectrum by analyzing the obtained skin spectrum and by determining a degree of distortion of the obtained skin spectrum in 1020.

For example, the bio-information estimation apparatus 700 may determine the degree of distortion of the obtained skin spectrum based on at least one from among the following: a light intensity of the obtained skin spectrum; absorbance for a wavelength; a spectrum reconstructed based on a skin composition spectrum; and similarity between the reference skin spectrum and the obtained skin spectrum.

Further, the bio-information estimation apparatus 700 may generate a guide for correcting at least one of a contact state and a measurement position of a spectrum obtainer with respect to the subject according to the degree of distortion of the obtained skin spectrum, and may output the generated guide by using at least one of an acoustic method, a visual method, and a tactile method.

The bio-information estimation apparatus 700 may estimate bio-information by using a bio-information correlation model pre-generated based on a valid skin spectrum in 1030.

For example, the bio-information estimation apparatus 700 may select an appropriate bio-information correlation model according to the types of bio-information to be measured, and may measure bio-information by comparing the selected bio-information correlation model with the obtained skin spectrum.

The disclosure can be provided in a computer-readable code written on a computer-readable recording medium. Codes and code segments provided by the disclosure can be easily deduced by computer programmers of ordinary skill in the art. The computer-readable recording medium may be any type of recording device in which data is stored in a computer-readable manner. Examples of the computer-readable recording medium include a ROM, a RAM, a CD-ROM, a magnetic tape, a floppy disc, an optical disk, and the like. Further, the computer-readable recording medium can be distributed over a plurality of computer systems connected to a network so that a computer-readable recording medium is written thereto and executed therefrom in a decentralized manner.

At least one of the components, elements, modules or units represented by a block as illustrated in the drawings may be embodied as various numbers of hardware, software and/or firmware structures that execute respective functions described above, according to an embodiment. For example, at least one of these components, elements or units may use a direct circuit structure, such as a memory, a processor, a logic circuit, a look-up table, etc. that may execute the respective functions through controls of one or more microprocessors or other control apparatuses. Also, at least one of these components, elements or units may be specifically embodied by a module, a program, or a part of code, which contains one or more executable instructions for performing specified logic functions, and executed by one or more microprocessors or other control apparatuses. Also, at least one of these components, elements or units may further include or be implemented by a processor such as a central processing unit (CPU) that performs the respective functions, a microprocessor, or the like. Two or more of these components, elements or units may be combined into one single component, element or unit which performs all operations or functions of the combined two or more components, elements of units. Also, at least part of functions of at least one of these components, elements or units may be performed by another of these components, element or units. Further, although a bus is not illustrated in the above block diagrams, communication between the components, elements or units may be performed through the bus. Functional aspects of the embodiments may be implemented in algorithms that execute on one or more processors. Furthermore, the components, elements or units represented by a block or processing steps may employ any number of related art techniques for electronics configuration, signal processing and/or control, data processing and the like.

Although a few embodiments have been shown and described, it would be appreciated by those skilled in the art that changes may be made in exemplary embodiments without departing from the principles and spirit of the disclosure, the scope of which is defined in the claims and their equivalents.

What is claimed is:

1. A spectrum quality assessment apparatus, comprising:
a spectrum obtainer configured to obtain a skin spectrum; and
a processor configured to assess a quality of the obtained skin spectrum based on at least one of a light intensity of the obtained skin spectrum, an absorbance with respect to a wavelength of the obtained skin spectrum, a spectrum reconstructed based on a skin composition spectrum, and a similarity between a reference skin spectrum and the obtained skin spectrum.

2. The apparatus of claim 1, wherein the obtained skin spectrum comprises a skin near-infrared absorption spectrum.

3. The apparatus of claim 1, wherein in response to the light intensity in at least one from among a wavelength region of 4000 cm⁻¹ to 4150 cm⁻¹ and a wavelength region of 4960 cm⁻¹ to 5280 cm⁻¹ of the obtained skin spectrum exceeding a predetermined threshold, the processor is configured to determine that the obtained skin spectrum is distorted.

4. The apparatus of claim 1, wherein the processor is configured to obtain a water path length based on a change in the absorbance with respect to the wavelength of the obtained skin spectrum and a reference absorbance with respect to a wavelength of a spectrum predetermined according to a center distance between a light source and a light detector, and in response to the obtained water path length being less than a predetermined value, the processor is configured to determine that the obtained skin spectrum is distorted.

5. The apparatus of claim 1, wherein the processor is configured to reconstruct the spectrum based on the skin composition spectrum, and in response to a difference in the absorbance between the obtained skin spectrum and the reconstructed spectrum exceeding a predetermined threshold, the processor is configured to determine that the obtained skin spectrum is distorted.

6. The apparatus of claim 5, wherein the processor is configured to generate the reconstructed spectrum by using a Classical Least square (CLS) algorithm.

7. The apparatus of claim 1, wherein the processor is configured to obtain the similarity between the obtained skin spectrum and the reference skin spectrum according to a change of the absorbance with respect to the wavelength, and in response to the similarity being less than a predetermined threshold, the processor is configured to determine that the obtained skin spectrum is distorted.

8. The apparatus of claim 1, wherein the spectrum obtainer comprises a light source configured to emit light to a subject, and a light detector configured to detect light reflected or scattered from the subject.

9. The apparatus of claim 7, further comprising an output part configured to output at least one from among a result of quality assessment of the obtained skin spectrum and guide information to correct distortion of the obtained skin spectrum.

10. A bio-information estimation apparatus, comprising:
a spectrum obtainer comprising a light source configured to emit light to a subject, and a light detector configured to detect light reflected or scattered from the subject; and
a processor configured to determine a degree of distortion of an obtained skin spectrum, and to estimate bio-information based on a valid skin spectrum determined according to the degree of distortion.

11. The apparatus of claim 10, wherein the processor is configured to determine the degree of distortion of the obtained skin spectrum based on at least one from among a light intensity of the obtained skin spectrum, an absorbance with respect to a wavelength, a spectrum reconstructed based on a skin composition spectrum, and a similarity between a reference skin spectrum and the obtained skin spectrum.

12. The apparatus of claim 11, wherein in response to determining that the obtained skin spectrum is not valid according to the degree of distortion, the processor is configured to control the spectrum obtainer to re-obtain a skin spectrum.

13. The apparatus of claim 12, wherein the processor is configured to generate guide information to correct at least one from among a state of a contact between the spectrum obtainer and the subject and a measurement position of the spectrum obtainer with respect to the subject according to the degree of distortion of the obtained skin spectrum.

14. The apparatus of claim 10, wherein the processor is configured to estimate bio-information by using a bio-information correlation model pre-generated based on the valid skin spectrum.

15. A spectrum quality assessment method, comprising:
obtaining a skin spectrum; and
assessing a quality of the obtained skin spectrum based on at least one from among a light intensity of the obtained skin spectrum, an absorbance with respect to a wavelength of the obtained skin spectrum, a spectrum reconstructed based on a skin composition spectrum, and a similarity between a reference skin spectrum and the obtained skin spectrum.

16. The method of claim 15, wherein the obtained skin spectrum comprises a skin near-infrared absorption spectrum.

17. The method of claim 15, wherein the assessing comprises determining that the obtained skin spectrum is distorted in response to the light intensity in at least one from among a wavelength region of 4000 cm⁻¹ to 4150 cm⁻¹ and a wavelength region of 4960 cm⁻¹ to 5280 cm⁻¹ of the obtained skin spectrum exceeding a predetermined threshold.

18. The method of claim 15, wherein the assessing comprises:
obtaining a water path length based on a change in the absorbance with respect to the wavelength of the obtained skin spectrum and a reference absorbance with respect to a wavelength of a spectrum predetermined according to a center distance between a light source and a light detector; and
in response to the water path length being less than a predetermined value, determining that the obtained skin spectrum is distorted.

19. The method of claim 15, wherein the assessing comprises:
generating the spectrum reconstructed based on the skin composition spectrum; and
in response to a difference in the absorbance between the obtained skin spectrum and the reconstructed spectrum exceeding a predetermined threshold, determining that the obtained skin spectrum is distorted.

20. The method of claim 15, wherein the assessing comprises:
obtaining the similarity between the obtained skin spectrum and the reference skin spectrum according to a change of the absorbance with respect to the wavelength; and
in response to the similarity being less than a predetermined threshold, determining that the obtained skin spectrum is distorted.
